(19) United States
(12) Patent Application Publication
(10) Pub. No.: US 2026/0256868 A1
(43) Pub. Date: Sep. 3, 2026

(54) COMBINATION HERBAL MEDICINES FOR THE TREATMENT OF INFLAMMATORY BOWEL DISORDERS

(71) Applicants: EVINATURE LTD., Binyamina (IL); SHEBA IMPACT LTD., Ramat-Gan (IL)

(72) Inventors: Shomron Silan Ben-Horin, Ramat Efal (IL); Nir Salomon, Binyamina (IL)

(21) Appl. No.: 18/880,109

(22) PCT Filed: Jun. 28, 2023
(86) PCT No.: PCT/IL2023/050662

§ 371 Date: Dec. 30, 2024

Related U.S. Application Data
(60) Provisional application No. 63/357,317, filed on Jun. 30, 2022.
(60) Provisional application No. 63/485,358, filed on Feb. 16, 2023.

(51) Int. Cl. A61K 36/9066; A61K 9/00; A61K 9/28; A61K 9/48; A61K 36/195; A61K 36/315; A61K 36/48; A61P 1/04

(52) U.S. Cl. CPC A61K 36/9066; A61K 9/0053; A61K 9/28; A61K 9/4816; A61K 9/4891; A61K 36/195; A61K 36/315; A61K 36/48; A61P 1/04

(57) ABSTRACT

The present invention is directed to combination therapies using compositions of Curcumin and Qing-Dai or extracts of plants producing thereof, and their clinical applications to the treatment of inflammatory conditions in general and to Inflammatory Bowel Diseases (IBD) in particular, including Crohn's disease, ulcerative colitis and related clinical conditions.

### TECHNOLOGICAL FIELD

The invention pertains to the field of herbal medicines and actives derived from such medicines, and their applications to inflammatory conditions in general and to Inflammatory Bowel Diseases (IBD) in particular, including Crohn's disease, ulcerative colitis and related clinical conditions.

### BACKGROUND

Ulcerative colitis (UC) is an intractable inflammatory bowel disease (IBD) which incidence is rapidly increasing worldwide, most prominently in the US and Japan. The conventional therapies for IBD predominantly rely on the use of 5-aminosalicylic acid (5-ASA), steroids, and certain types of immunomodulators (e.g., azathioprine, 6-methyl-mercaptopurine) delivered under specific dose regimens and modes of administration. In the recent years, alternative remission inducing therapies have been introduced, using biologics such as monoclonal antibodies (e.g., infliximab, adalimumab, ustekinumab, and vedolizumab), low molecular weight compounds (e.g., tofacitinib) and calcineurin inhibitors (e.g., cyclosporine and tacrolimus). Despite the reported efficacy of these treatments, long-term management of IBD still poses a significant challenge, especially in intractable patients who are resistant to these treatments or become resistant with time, or groups who are treatment intolerant, such as pregnant women or patients with allergies, for example. Some natural products have been suggested as optional substitutes in these conditions, but their actual contribution to treatment efficacies remains undecided.

Traditional herbal medicines and herbal extracts have been used for centuries for treating and everyday management of chronic inflammatory conditions. Only a few of them, however, have evidence-based proof of clinical efficacy, in general, and in IBD in particular. Specific examples are Chinese herbal medicine Qing-Dai (QD), also known as Indigo Naturalis (IN), and Curcumin. Both have been tested alone in several studies, predominantly as maintenance therapies in combination with conventional drugs for active IBD [1-10]. Together with this, some studies reported significant adverse reactions with prolonged use of QD, especially in high doses [11].

### REFERENCES

1. Hanai H et al. Curcumin maintenance therapy for ulcerative colitis: randomized, multicenter, double-blind, placebo-controlled trial. Clin Gastroenterol Hepatol 2006, 4 (12): 1502-1506.
2. Singla V et al. Induction with NCB-02 (curcumin) enema for mild-to-moderate distal ulcerative colitis-A randomized, placebo-controlled, pilot study. J Crohn's Colitis 2014, 8 (3): 208-214.
3. Lang A et al. Curcumin in combination with mesalamine induces remission in patients with mild-to-moderate ulcerative colitis in a randomized controlled trial. Clin Gastroenterol Hepatol 2015, 13 (8): 1444-9e1.
4. Sugimoto S et al. Clinical efficacy and safety of oral Qing-Dai in patients with ulcerative colitis: A single-center open-label prospective study. Digestion 2016, 93 (3): 193-201.
5. Wang J et al. Curcumin improves intestinal barrier function: modulation of intracellular signalling, and organization of tight junctions. Am J Physiol Cell Physiol 2017, 312 (4): C438-C445.
6. Kawai S al. Indigo Naturalis ameliorates murine dextran sodium sulfate-induced colitis via aryl hydrocarbon receptor activation. J Gastroenterol 2017, 52 (8): 904-919.
7. Naganuma M et al. Efficacy of indigo naturalis in a multicenter randomized controlled trial of patients with ulcerative colitis. Gastroenteroly 2018, 154 (4): 935-947.
8. Uchiyama K et al. Efficacy and safety of short-term therapy with indigo naturalis for ulcerative colitis: An investigator-initiated multicenter double-blind clinical trial. PLOS One 2020, 15 (11): e0241337.
9. Saiki J P et al. Treatment-refractory ulcerative colitis responsive to indigo naturalis. BMJ Open Gastroenterol 2021, 8 (1): c000813.
10. Banerjee R et al. Novel bioenhanced curcumin with mesalamine for induction of clinical and endoscopic remission in mild-to-moderate ulcerative colitis: A randomized double-blind placebo-controlled pilot study. J Clin Gastroenterol 2021, 55 (8): 702-708.
11. Naganuma M et al. Adverse events in patients with ulcerative colitis treated with indigo naturalis: A Japanese nationwide survey. J Gastroenterol 2019, 54 (10): 891-896.

### GENERAL DESCRIPTION

The incentive behind the invention has been to find an effective, convenient, and safe alternative for treating chronic inflammatory systemic diseases (CIDs), which are common life-long debilitating disorders that are subject to increased mortality and high-cost maintenance therapies. According to current views, CID is at the root of diabetes, cardiovascular diseases, arthritis, neurodegenerative disorders and cancer, as well as various types of autoimmune disorders, chronic pulmonary conditions, and allergies. Altogether, today they are considered the leading cause of death worldwide.

As a proof of concept, the inventors have chosen IBDs, which is a group of chronic inflammatory disorders of the gastrointestinal (GI) tract, especially in view of that some of the IBDs, such as Crohn's disease (CD) and ulcerative colitis (UC), are intractable and resist conventional treatments. With active UC, for example, despite an increasing arsenal of new drugs such as biologics and small molecules, some patients remain refractory to both types of treatment and others report significant adverse events of immune-suppressive therapies. Another problem with these new drugs is very high costs and their limited availability in some countries.

Herbal medicines can generally offer safe and convenient alternatives for long term management of chronic conditions. The question remains whether they can be effective with proven demonstrable short-term benefits for acute clinical conditions such as active UC. And further, whether specific combinations of herbal medicines with specific proportions of active components and/or under specific dose regimens may have enhanced therapeutic effects. And ultimately, whether such combinations of herbal medicines can replace or minimize the use of the currently recognized medicines and their related adverse reactions.

According to the present invention, a combination of two herbal extracts containing Curcumin (Cur) and Qing-Dai (QD) has proved to be successful in all these respects, at least for the two main intractable IBDs, CD and active UC, and potentially for other CIDs.

Both Cur and QD are known herbal medicines and have been already approved as herbal supplements in some countries (e.g., Israel). Cur or QD alone have been implemented to some extent, always as add-ons, to anti-inflammatory and/or immunosuppression therapies, as maintenance therapies for active UC. According to the present invention, a combination Cur and QD (CurQD), when administered orally under specific dose regimens and tapering procedure, alone or as add-on to routine therapies, can be effective and safe for treating intractable active UC by a number of clinical and inflammatory indices of UC and lack of observable side effects. By the present examples, observable curative effects of oral CurQD in powder form became apparent within weeks from initiation of the CurQD therapy and persisted through the entire study period with the continued use of the CurQD protocol.

These results were surprising in view of the known precautions with the oral use of QD, especially in powder form. It was previously reported that oral use of single herbal powder preparations of QD was related to significant side effects, typically including diarrhea, abdominal pain, nausea, vomiting, liver dysfunction, cutaneous symptoms, leukocyte decrease, dizziness, headache and pulmonary hypertension [Zhou D et al in Eur J Integr Med 2014, 6:135-146; Ferber K H in J Environ Pathol Toxicol Oncol 1987, 7:73-83]. In other words, the specific CurQD combinations and dose regimens of the invention have succeeded not only to exceed the anti-inflammatory effects of Cur and QD alone and potentially supersede the existing IBD therapies, but also to resolve the problem of QD toxicity. Furthermore, the present examples have demonstrated that the proposed CurQD therapy can be equally successful for the adult and pediatric UC. Therefore, when considering potential applications and in view of the known diagnostic overlap between UC and CD, it can be presumed with a degree of confidence that the proposed approach can be further successful for CD and other IBDs, and potentially for other types of CIDs.

More specifically, the feasibility of short-term treatment with CurQD was tested in a preliminary case study of two patients with moderate-to-severe UC. Patient 1, a 24 years-old male, presented with symptoms of severe UC, including severe mucosal inflammation (Mayo 3) and progressive worsening of symptoms, which was further refractory to cyclosporine and corticosteroids, had only partial response to infliximab and responded to infliximab and 6-mercaptopurine (6-MP). Daily oral administering of 2.5 gr CurQD powder (1000 mg Cur and 1500 mg QD) in this patient led to rapid cessation of rectal bleeding and complete clinical remission after few weeks. Follow-up endoscopies (12 weeks) indicated only minimal residual inflammation. After infliximab was terminated due to reimbursement issues, the patient was successfully maintained on tapered doses of CurQD (1000 mg Cur and 500 mg QD) and 6-MP for 31 months. Two intermittent flares were treated by reversal to initial doses of CurQD (1000 mg Cur and 1500 mg QD). EXAMPLE 1

Patient 2, a 59 years-old female, presented with severe UC, severe mucosal ulceration by endoscopy (Mayo 3), which was refractory to oral 5-ASA and corticosteroids (the patient refused the recommendation for biologics). Daily oral administering of 2 gr CurQD powder (1000 mg Cur and 1000 mg QD) led to rapid clinical remission as evidenced by lower endoscopies after 2 months on CurQD, and to complete mucosal healing after 5 months on CurQD. The patient maintained clinical remission with tapered doses of CurQD (1000 mg Cur and 500 mg QD) as the only treatment for at least 49 months. None of the patients showed noticeable adverse events. EXAMPLE 1

In a further prospective study, patients diagnosed with active UC by clinical and inflammatory indices of UC (N=10) received daily oral doses of 3 gr CurQD powder (3×500 mg Cur and 3×500 mg QD) in enteric-coated capsules (n=3) for the period of 4 weeks. Concomitant therapies with biologics or cortico-steroids were allowed before and during the study. Success of the CurQD therapy was assessed by Simple Clinical Colitis Index (SCCAI) and Fecal Calprotectin (Fcal). Overall, about 60% of the patients showed significant clinical response to CurQD (reduction of SCCAI≥3), about 40% showed significant biomarker response (>50% Fcal reduction) and about 30% achieved clinical remission (SCCAI≤2). EXAMPLE 2

These results were further substantiated by a subsequent double-blind placebo-controlled trial of patients with active UC (N=42), receiving either daily oral doses of 3 gr CurQD powder (as above) or placebo for the period of 8 weeks, with or without concomitant medications (as above). This study revealed significant differences between the CurQD and placebo groups by a number of clinical outcomes, including therapy response (reduction in SCCAI≥3) and objective evidence improvement (Mayo endoscopic sub-score change ≥1 or Fcal reduction >50% from baseline) as the co-primary outcomes, and clinical remission (SCCAI≤2) at week 8 as the secondary outcome. The co-primary outcomes were achieved in 43% vs. 8% patients (p=0.033) and the secondary outcome in 50% vs. 8% patients (p=0.01), for CurQD and placebo groups, which was further supported by the specific outcomes of therapy response in 85.7% vs. 30.7% patients (p<0.001), endoscopic improvement in 75% vs. 20% patients (p=0.036) and >50% Fcal reduction in 46.4% vs. 15.4% (p=0.08) patients, respectively. Overall, this study reinforced the notion that oral CurQD therapy has a prospect of providing an effective solution, either as add-on or as a replacement therapy, for the treatment of UC and potentially for other IBDs. EXAMPLE 3

In the course of time, the inventors developed a more advanced dosing protocol by tapering the relative amounts or weight ratios of QD in the CurQD combination, thereby maximizing the efficacy of the oral CurQD therapy and minimizing or significantly reducing the risk of side effects of QD. One example of such protocol is a temporally tailored protocol using incrementally decreasing doses or weight ratios of QD in the CurQD combination in the course of treatment, starting (e.g., first 4-8 weeks) from higher daily doses or weight ratios of QD vs. Cur (e.g., 1-2 gr QD vs. 1-2 gr Cur) as a short-term treatment in active phase of the disease and continuing with gradually tapered (e.g., every two weeks) daily doses or weight ratios of QD vs. Cur (e.g., to 1.5, 1.2, 1, 0.8, 0.6 and 0.5 gr QD vs. 2, 2.2 and 2.5 gr Cur), and ending with daily doses of Cur without QD (e.g., 2-3 gr Cur) as long-term treatment for the maintenance of remission. Upon emergence of clinical flare, the protocol reverts to the short-term CurQD dosing as above. The temporally tailored protocol has already been proved efficient and safe in the treatment of active UC and is now tested in other IBDs and CIDs. EXAMPLE 4

The success of oral Cur QD therapy for the treatment of IBD, and UC in particular has received additional support in two more recent studies. One was a retrospective multicenter cohort study of adult UC patients (N=88), showing that oral CurQD therapy leads to a significant clinical response (SCCAI≥3 reduction from baseline) in about 61% of the patients and clinical remission (SCCAI≤2 and ≥3 points reduction from baseline) in about 47% of the patients, and by the Fcal biomarker can lead to the biomarker response (≥50% Fcal reduction from baseline) in about 88% of the patients and the biomarker remission (Fcal≤100 μg/g at the end of induction) in about 62% of the patients. It should be noted that the oral CurQD therapy was used either alone or as add-on, about half of the patients further received biologics and/or small molecules or other new-generation medications. EXAMPLE 5

Another study was a retrospective cohort study of pediatric patients with mild-to-moderate UC (N=30), confirming that the oral CurQD therapy can be further effective and safe also in pediatric patients, and especially when biologics and other conventional drug proved to be ineffective. According to this relatively small study, the oral CurQD therapy could provide an adequate therapy response and remission in the majority of patients with relatively low side effects that could be attributed to QD. This approach is now tested under the temporally tailored dosing protocol in a more extended prospective study of pediatric patients with UC. EXAMPLE 6

The inventors could further show that the use of CurQD combination permitted to reduce the concentrations of Indigo and Indirubin, the two active components responsible for the majority of QD related side effects, below the critical risk conferring threshold. The effective concentrations of the two actives could be reduced from 2% Indigo and 0.13% Indirubin, the gold standard according to the Chinese Pharmacopoeia (Qi-Yue Y et al in Chin Med 2020, 15 (1): 127), to as low as 0.55% Indigo and 0.06% Indirubin while retaining sufficient therapeutic efficacy by several IBD parameters. Moreover, the safety profile could be further improved by concomitant use of the temporally tailored dosing protocol, revealed by total absence of pulmonary hypertension events and neither finding of elevated pulmonary vascular pressure in patients treated with this CurQD combination as opposed to the recorded rate of pulmonary hypertension of 1:90-100 in patients treated with QD alone (Naganuma, J in Gastroenterol 2019 54 (10): 891-896). EXAMPLE 7

More recently, the inventors explored the mechanism of action of CurQD and its downstream effectors. Several studies suggested that QD is a putative agonist of the aryl-hydrocarbon receptor (AhR) and thus acting as an activator of the AhR signaling pathway (Guengerich et al in Arch Biochem Biophys 2004, 423:309-316; Kawai et al in J Gastroenterol 2017, 52:904-919; Adachi et al in J Biol Chem 2001, 276:31475-8). AhR activation has been further implicated in the amelioration of UC and CD. AhR is essentially a ligand-activated transcription activator of various genes, including among others cytochrome P450 1A1 (CYP1A1) which is an important xenobiotic-metabolizing enzyme and an important regulator of immune response in the intestine.

Analysis of CYP1A1 expression levels in colonic specimens obtained from UC patients treated with oral CurQD and patients treated by other medications or placebo revealed that CYP1A1 were significantly upregulated patients receiving the oral CurQD therapy, and further that this phenomenon was absent in the placebo and other treatments (5ASA or biologics) and was highly specific CurQD. Therefore, for being distinct form other IBD drugs and operating via another molecular mechanism, CurQD can provide a novel and promising approach to the treatment of IBDs and other inflammatory conditions in recruiting a complementary pathway and a series of additional effectors that could contribute to the curative effects of CurQD. In other words, these findings suggest that the effects of CurQD can be further enhanced by the addition of other effectors and activators of the AhR signaling pathway. Examples of such activators are various AhR ligands that can be found in arachidonic acid-(ARA), tryptophan- or flavonoid-rich diets and supplements, yogurt, vitamins, probiotics and prebiotics, etc. EXAMPLE 8

Altogether, these studies provide proof of concept for the applicability of the oral CurQD therapy for IBDs, and potentially for other CIDs. They further suggested a relatively high compliance to the oral CurQD therapy in adult and pediatric patients and a good likelihood of incorporating it into the patients' daily routine. They further showed that the oral CurQD therapy can be equally successful as add-on and as single drug therapy in providing short-term and long-term treatment of acute and chronic IBD and further in preventing or reducing recurrent IBD episodes. Still further, they showed that the curative effects of the oral CurQD therapy can be further enhanced by using QD extracts with lower content of actives and temporally tailored dosing regiments that improve therapeutic efficacy of the CurQD combination while reducing or minimizing the side effects of QD.

More broadly, the presently proposed inventive concept can serve as a basis for design and development of a series of drug products, dosage forms, methods and kits using Cur and QD in various relative amounts and weight ratios, per se or in combination with other actives, which can be formulated or adapted for oral or enteric administrations so as to provide short- and long-term treatments in adult and pediatric patients with acute and chronic IBD. Living with IBD is a continuous challenge to keep the right balance between therapy efficacy and its adverse effects that are ultimately reflected in drug response and patient's compliance. Therapies using natural-based actives that are generally considered as safe (GRAS) may provide alternative, more manageable and individually adjusted solutions for highly burdensome and nonetheless common chronic conditions, leading to a better patient's compliance and a better quality of life.

### BRIEF DESCRIPTION OF THE DRAWINGS

Certain embodiments of the invention are described by way of examples with reference to the following figures.

FIGS. 1a-1b illustrate the effect of the oral CurQD therapy in the preliminary case study of two patients with active UC. Figures illustrate the use of consecutive lower endoscopy images as indicators of UC improvement, with Patient 1 receiving the therapy as add-on (FIG. 1a, at T₀ and after 2 and 3 months) and Patient 2 as a single therapy (FIG. 1b, at T₀ and after 2 and 7 months.

FIGS. 2a-2b show the effect of the oral CurQD therapy in the prospective study of patients with active UC receiving the therapy as add-on. Figures illustrate the use of two important clinical indicators UC improvement, Simple Clinical Colitis Index (SCCAI) (FIG. 2a) and Fecal Calprotectin (FIG. 2b), both showing significant reduction in the treated patients at the end of the study period (4 weeks).

FIG. 3 illustrates certain oral dosage forms of the invention that were used in the double-blind controlled trial. The capsules (CurQD and placebo) contain therapeutic amounts of Cur and QD in powder form encapsulated in enteric coating.

FIG. 4 shows the effect of the oral CurQD therapy in the double-blind controlled trial of patients with active UC receiving CurQD as add-on or placebo. Figure illustrate the use of additional markers of UC improvement, such as clinical response (reduction in SCCAI≥3), biomarker response (>50% Fcal reduction from baseline) and endoscopic response (Mayo endoscopic sub-score change≥1), and the sum total of these markers (co-primary outcomes), and clinical remission (SCCAI≤2), all showing significant reduction in the CurQD treated group (grey) vs. placebo (black) at the end of study period (8 weeks).

FIGS. 5a-5b illustrate the use of specific marker of UC improvement in the same trial, i.e., Fcal levels, showing significant reduction (median Fcal levels) at the end of study period (8 weeks) in the CurQD group (FIG. 5a) vs. placebo (FIG. 5b) (p<0.001).

FIG. 6 illustrates the use of the same markers in the retrospective multicenter cohort study of patients with active UC, showing improvement of UC by clinical response (SCCAI≥3 reduction from baseline) in 61.4%, clinical remission (SCCAI≤2 and ≥3 points reduction from baseline) in 46.6%, biomarker (FC-Fcal) response (≥50% reduction from baseline) in 87.9% and biomarker remission (FC≤100 μg/g at the end of induction) in 62.15% of the patients subjected to the oral CurQD therapy.

FIGS. 7a-7c illustrate the use of specific markers of UC improvement in the same study at T₀ and at the primary endpoint (8-12 weeks), showing significant reduction in the median SCCAI scores (FIG. 7a, p<0.0001) and reduction in the median FC levels (FIG. 7b, log FC p<0.0001), and FC raw data (FIG. 7c).

FIGS. 8a-8b illustrate the use of additional marker of inflammation, the C-reactive protein (CRP), in the same study, showing significant reduction of CRP levels (FIG. 8a, median CRP p<0.0007), and CRP raw data (FIG. 8b) at end of study period (8-12 week).

FIGS. 9a-9b illustrate methods for determining Indigo and Indirubin concentrations in preparations of QD and CurQD: in FIG. 9a (i) Extracted Ion Chromatogram (EIC) with deprotonated precursor ions Indigo (260.060 m/z, [M−H]⁻) and radical anion Indirubin (261.068 m/z, [M]), in FIG. 9a (ii) UV chromatogram (detection at 611 nm) of Indigo and Indirubin (x-axis represents retention time and γ-axis signal intensity), and in FIG. 9b Fragmentation Mass Spectra by collision-induced dissociation of Indigo and Indirubin (x-axis represents mass to charge (m/z) and γ-axis signal intensity, precursor ions labeled ♦). Putative assignments of characteristic fragment ions are shown.

FIGS. 10a-10b show the effect of CurQD therapy on the expression of cytochrome P450 1A1 (CYP1A1), an important xenobiotic-metabolizing enzyme and an important regulator of immune response in the intestine. Figures show that patients receiving the oral CurQD therapy had elevated levels of CYP1A1 (FIG. 10a), which was specific to CurQD as it was absent in placebo (FIG. 10a) and other treatments using 5ASA or biologics (FIG. 10b), suggesting that CurQD acts through an alternative mechanism involving the aryl-hydrocarbon receptor (AhR).

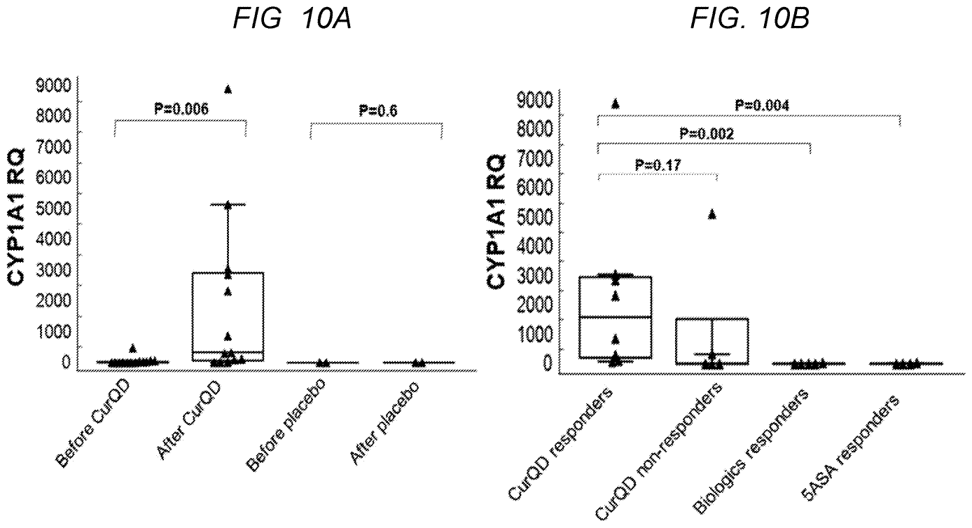

### DETAILED DESCRIPTION OF EMBODIMENTS

One of the central objectives of the invention was to provide natural-based compositions with anti-inflammatory properties that can offer satisfactory alternative for analogous chemically based drugs without the associated risks of adverse events (e.g., cardiovascular events associated with many anti-inflammatory drugs). To that end, the inventors have found that certain combinations of Curcumin (Cur) and Qing Dai (QD) posses improved anti-inflammatory properties that surpass the properties of the individual Cur and QD components and are free of side effects that are often attributed to QD alone.

Cur and QD are two natural substances that are produced by some plants. Both have been associated with strong anti-inflammatory properties and as such have been extensively used in indigenous or folk medicine. QD per se has been associated with significant adverse reactions, including diarrhea, abdominal pain, nausea, vomiting, liver dysfunction, cutaneous symptoms, leukocyte decrease, dizziness and headache, and further pulmonary hypertension.

Thus, in the broadest sense the compositions of the invention can be described as compositions comprising a combination of Cur and QD, or an extract of any part of a plant producing Cur or QD, wherein Cur and QD are present in specific proportions or weight ratios, depending on the desired effect and intended use.

In some embodiments the composition can comprise a combination wherein the Cur and QD are present in a weight ratio ranging between 100,000:1 and 1:100,000, respectively.

The term “Curcumin” (Cur) predominantly refers herein to the principal curcuminoid of turmeric and the term “Qing-Dai” (QD) the herbal medicine under that name, also known as Indigo naturalis.

More specifically, the term Cur encompasses herein a range of natural-based substances comprising a compound with a general molecular formula C₂₁H₂₀O₆ or a general structure of:

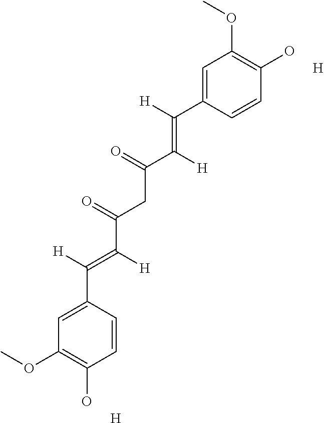

It further encompasses substances comprising the compound referred to as 1,7-bis(4-hydroxy-3-metho-xyphenyl)-1,6-heptadiene-3,5-dione, or diferuloylmethane, which is the main natural polyphenol found in the rhizome of Curcuma longa (turmeric) and in other Curcuma spp and members of the ginger family, Zingiberaceae. It further implies herein a bright yellow chemical produced by this family of plants. It encompasses any extracts of plants containing these types of compounds and any derivatives thereof, and further mixes and combinations of substances comprising thereof. It further encompasses a variety of herbal supplements sold by this name, including pure curcumin.

In some embodiments Cur can be provided in the form of a powder.

In some embodiments Cur can be provided in the form of an extract, a solution or a formulation for enhanced absorption.

The term “extract” encompasses herein a range of preparations extracted from turmeric plants, including ethanolic extracts of turmeric, water extracts or polar extracts of turmeric rich in polysaccharides and oil extracts (also turmeric essential oil).

In some embodiments Cur extracts can be standardized to include 80-95% curcuminoids, primarily curcumin.

The term “Qing-Dai” (QD), also Indigo Naturalis (IN) or Indigofera Tincturia (IT), generally refers herein to blue pigment extracted from the leaves and stems of plants such as Assam indigo (Strobilanthes cusia of the Acanthaceae family), false indigo (Indigofera bungeana Walp of the Fabaceae family), and woad (Isatis tinctoria of the Brassicaceae family). It further refers to the Chinese medicine by that name extracted from this type of plans which is characterized by certain content of natural ingredients such as Indigo, Indirubin, Isoindigotin, and Nimbosterol.

Indigo is essentially a mix of Indigo/Indirubin/Isoindigotin with a general molecular formula C₁₆H₁₀N₂O₂ or a general structure of one of:

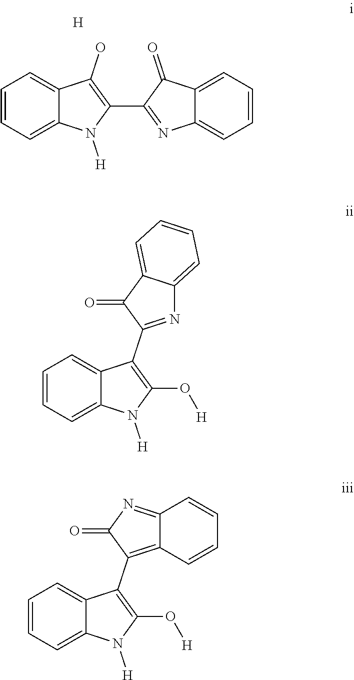

In some embodiments the compositions of the invention can comprise the natural active ingredients of QD extracts, i.e., Indigo, Indirubin, Isoindigotin, Nimbosterol, or any combination thereof.

In some embodiments the QD extracts can comprise Indigo and Indirubin at specified concentrations and proportions. In some embodiments the QD extracts can comprise Indigo and Indirubin at concentrations of at least 2% and at least 0.13% per total weight of the QD extract (w/w), which is the gold standard of the Chinese Pharmacopeia. (Qi-Yue et al. Chin Med 2020 15 (1): 127).

In some embodiments the QD extracts can comprise Indigo and Indirubin at concentrations other than 2% and 0.13% (w/w), respectively.

In some embodiments the QD extracts can comprise Indigo at a concentration in the range between 0.1% and 6% (w/w) and Indirubin at a concentration in the range between 0.01% and 1% (w/w), or more specifically Indigo at a concentration in the range between 0.1%-0.2%, 0.2%-0.3%, 0.3%-0.4%, 0.4%-0.5%, 0.5%-0.6%, 0.6%-0.7%, 0.7%-0.8%, 0.8%-0.9%, 0.9%-1%, 1%-1.5%, 1.5%-2%, 2%-2.5%, 2.5%-3%, 3%-3.5%, 3.5%-4%, 4%-4.5%, 4.5%-5%, 5%-5.5%, 5.5%-6% (w/w) and Indirubin at a concentration in the range between 0.01%-0.02%, 0.02%-0.03%, 0.03%-0.04%, 0.04%-0.05%, 0.05%-0.06%, 0.06%-0.07%, 0.07%-0.08%, 0.08%-0.09%, 0.09%-0.1%, 0.1%-0.2%, 0.2%-0.3%, 0.3%-0.4%, 0.4%-0.5%, 0.5%-0.6%, 0.6%-0.7%, 0.7%-0.8%, 0.8%-0.9%, 0.9%-1% (w/w).

In some embodiments the QD extracts can comprise Indigo at a concentration of about 0.5% to about 1% (w/w) and Indirubin at a concentration of about 0.05% to about 0.1% (w/w).

In some embodiments QD extracts or the respective active ingredients can be obtained from Strobilanthes cusia, Indigofera bungeana and Isatis tinctoria.

In some embodiments QD extracts or the active ingredients can be obtained from Baphicacanthus cusia, (Acanthaceae) and Polygonum tinctorium (Polygonaceae).

The QD extracts which are applicable to the invention can be obtained from any part of these plants (roots, stems, leaves, flowers and fruits) using various methods, such as extraction in acetic ether or column extraction (e.g., Si gel column, alumina column).

In some embodiments the QD extracts can be provided in the form of a powder.

In some embodiments the QD extracts can be in liquid or liquid gel forms.

In some embodiments the QD extracts can be provided in the form of oils.

In some embodiments the Cur and QD extracts can further contain oral absorption enhancers. Examples of natural-based absorption enhancers are piperine, sodium caprate (medium-chain fatty acid found in milk) and sodium cholate (bile salt constituent).

Regarding the Cur and QD proportions in the compositions of the invention, in the broadest sense the compositions can comprise Cur and QD in a weight ratio ranging between 100,000:1 and 1:100,000 respectively. In other words, they can comprise a Cur: QD ratio ranging from 100,000:1, 90,000:1, 80,000:1, 70,000:1, 60,000:1, 50,000:1, 40,000:1, 30,000:1, 20,000:1, 10,000:1, 9,000:1, 8,000:1, 7,00:1, 6,000:1, 5,000:1, 4,000:1, 3,000:1, 2,000:1, 1,000:1, 900:1, 800:1, 700:1, 600:1, 500:1, 400:1, 300:1, 200:1, 100:1, 90:1, 80:1, 70:1, 60:1, 50:1, 40:1, 30:1, 20:1, 10:1 in favor of Cur (w/w) and up to 1:100,000, 1:90,000, 1:80,000, 1:70,000, 1:60,000, 1:50,000, 1:40,000, 1:30,000, 1:20,000, 1:10,000, 1:9,000, 1:8,000, 1:7,000, 1:6,000, 1:5,000, 1:4,000, 1:3,000, 1:2,000, 1:1,000, 1:900, 1:800, 1:700, 1:600, 1:500, 1:400, 1:300, 1:200, 1:100, 1:90, 1:80, 1:70, 1:60, 1:50, 1:40, 1:30, 1:20, 1:10 in favor of QD (w/w).

In some embodiments the compositions can comprise a Cur: QD ratio ranging from 10:1, 9:1, 8:1, 7:1, 6:1, 5:1, 4:1, 3:1, 2:1 in favor of Cur (w/w) and up to 1:10, 1:9, 1:8, 1:7, 1:6, 1:5, 1:4, 1:3, 1:2 in favor of QD (w/w).

In some embodiments the compositions can comprise a Cur: QD ratio of about 4:1, 7:3, 3:2, 3:1, 2:1, 1:1, 1:2, 1:3, 2:3, 3:7 or 1:4, respectively (w/w).

In other words, in some embodiments Cur can be provided in an excess (w/w).

In some embodiments QD can be provided in an excess (w/w).

In some embodiments Cur and QD can be provided in equal parts, or at the ratio of 1:1 (w/w).

In some embodiments the compositions can comprise very low or almost no QD.

In some embodiments Cur and/or QD can be provided in the form of powders.

In some embodiments Cur and/or QD can be provided in the form of liquid or oil extracts or liquid gel forms.

One of the important features of the present compositions is the ability to provide an improved therapeutic effect relative to the effect of the same amounts of Cur and QD when used alone and a reduced adverse effect relative to the same amount of QD alone. The term “therapeutic effect” predominantly refers to anti-inflammatory effects of Cur and QD, and the term “adverse effect” to the known adverse effects of QD.

Methods for measuring anti-inflammatory effects of actives or are well known in the art. They often involve imaging or laboratory analyses of the blood or serum. The laboratory analyses can involve measurements of neutrophil-to-lymphocyte ratio (NLR), lymphocyte-to-monocyte ratio (LMR), platelet-to-lymphocyte ratio (PLR), derived NLR (dNLR), neutrophil to lymphocyte, platelet ratio (NLPR), systemic inflammation index (SII), aggregate index of systemic inflammation (AISI), systemic inflammation response index (SIRI) and C-reactive protein-to-lymphocyte ratio (CRP/L). They can further involve clinical evaluation of severity of inflammation, the number and spread of inflammation sites and the recurrence of inflammation with the disease progression. Clinical evaluation is often assisted by imaging analyses.

Improvement of anti-inflammatory effect implies herein a positive change or a remission in one or more symptoms of inflammation, reduction in the number and/or spread of inflammation sites, reduction in the severity of symptoms and/or reduction in the recurrence of symptoms. It can further imply a shorter timeline to achieve each of these conditions.

Reduction of adverse effects of QD implies herein an absence or a reduction of at least one symptom of diarrhea, abdominal pain, nausea, vomiting, liver dysfunction, cutaneous symptoms, leukocyte decrease, dizziness, headache and/or the development of serious event of pulmonary hypertension, all of which were related to QD. Evaluation of adverse effects can further involve measurements of liver enzymes in the blood, i.e., ALP (alkaline phosphatase), ALT (alanine transaminase), AST (aspartate aminotransferase), and gamma-glutamyl transferase (GGT), by known in the art methods.

In some embodiments the anti-inflammatory effect of the compositions of the invention can be evaluated in the clinical context of a chronic systemic inflammatory disorder or condition (see below).

In some embodiments the anti-inflammatory effect of the compositions of the invention can be evaluated in the clinical context of IBD, using recognized clinical criteria, endoscopic imaging and laboratory tests (see below and EXAMPLES).

One advantage of the present compositions is in being adapted for enteral administration. The term “enteral” encompasses herein any type of administering that involves absorption via the GI tract, including oral, buccal, sublingual gastric, duodenal, and rectal administrations (e.g., enema or suppositories).

In numerous embodiments the compositions of the invention can be adapted to or formulated for oral administration.

In some embodiments the compositions can be compressed in a tablet, a pill or a capsule, with or without an enteric coating. Enteric coating encompasses herein any type of polymer that confers modified or controlled dissolution, adsorption or release actives (i.e., Cur and/or QD) into the gastric environment (e.g., stomach, duodenum, intestine or colon). The terms “controlled” or “modified” release in this context imply attenuated, sustained, prolonged or targeted release of actives.

In some embodiments the enteral coating can be made of one or more materials selected from EUDRAGIT® L or S polymers (e.g., EUDRAGIT® RL, RS and/or NM).

In some embodiments EUDRAGIT® can be modified with different acidic or alkaline end groups to allow for a specific pH-dependent release of the active ingredients in the stomach or the intestinal milieu.

It is another important objective of the invention to provide a series of pharmaceutical and nutraceutical products or compositions constructed on the basis of the aforementioned CurQD combinations and compositions and formulations comprising thereof.

In some embodiments the pharmaceutical nutraceutical compositions of the invention can further comprise a pharmaceutically acceptable carrier or excipient.

It is yet another objective of the invention to provide premade dosage forms comprising the compositions of the invention.

In some embodiments the dosage forms can be oral dosage forms.

In some embodiments the dosage forms can be in the form of suppositories or premade preparations for enema.

In numerous embodiments the dosage forms of the invention comprise preset amounts of Cur and QD that serve particular use or particular indication.

As a general rule, the dosage forms of the invention can comprise up to about 5000 mg Cur and up to about 5000 mg QD.

In other words, in some embodiments the dosage forms can comprise Cur at an amount of ranging between 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000 mg and QD at an amount of ranging between 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000 mg.

For specific applications the dosage from can comprise more than 5000 mg Cur and more than 5000 mg QD, or more specifically comprising Cur and QD in the amounts ranging between 5000-5500, 5500-6000, 6000-6500, 6500-7000, 7000-7500, 7500-8000, 8000-8500, 8500-9000, 9000-9500, 9500-10000 mg each, and more.

In some embodiments the dosage forms can comprise Cur at an amount ranging between 500-3000 mg and QD at an amount ranging between 500-3000 mg.

The amounts of Cur and QD in the dosage from can be different or the same.

In some embodiments the amount of Cur can be in the range between about 500 mg and about 2000 mg and the amounts of QD in the range between about 500 mg and about 2000 mg.

In some embodiments the amounts of Cur and QD can be different, or the amount of Cur can be in the range between about 500 mg and about 3000 mg and the amount of QD in the range between about 500 mg and about 2000 mg.

In some embodiments the amount of Cur can be in the range between about 500 mg and about 3000 mg and the amount of QD in the range between about 500 mg and about 1000 mg.

In some embodiments the amount of QD can be less than 500 mg, in other words, the amounts of Cur can remain in the range of between about 500 mg and about 3000 mg and the amount of QD can be in the range between about 1 mg and about 500 mg, or in the range of about 1-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-440, 440-450, 450-460, 460-470, 470-480, 480-490, 490-500 mg.

In some embodiments the dosage forms can comprise Cur and almost no QD.

The dosage forms with lower amounts of QD proved to be advantageous in the remission phase or for long-term maintenance of the patient.

To that end, the inventors have further devised a kit that provides the dosage forms of the invention with various amounts of Cur and QD, and instructions on how to apply specific dosage forms in different phases of the treatment protocol. For example, the kit can comprise a set of dosage forms with Cur and QD in equal amounts in the higher end of the specified ranges, and additional set(s) of dosage forms with the same or higher amounts of Cur and tapered amounts of QD.

In some embodiments the kit can comprise:
a set of dosage forms with Cur and QD at an amount of up to about 5000 mg each, or more,
one or more sets of dosage forms with Cur and tapered amounts of QD, a set of dosage forms with only Cur at an amount of up to about 5000 mg, and
instructions for use so as to account for treatments in the acute phase of the disease and the maintenance or remission phases.

The kit can be further personalized as per specific indication and patient's needs.

In some embodiments the kit and the dosage forms can be adapted for oral or rectal administration, including specific instructions for use and assisting devices.

One of the main applications of the presently proposed compositions, dosage forms and kits is for use in preventing, ameliorating, or treating chronic systemic inflammatory disorders or conditions (CIDs).

The term “Chronic Systemic Inflammatory Disorder” here implies a wide range of conditions, a major feature of which is a chronic inflammation in one or more body parts or tissues which is often reflected in elevated indices of inflammation in the blood. Notable examples of CIDs are diabetes, cardiovascular diseases, cancer, arthritis, neurodegenerative disorders, psychiatric disorders, autoimmune disorders, inflammatory skin disorders, chronic pulmonary conditions and allergies.

It is contemplated that the compositions, dosage forms and kits of the invention can be applied for the prevention, amelioration and treatment of a disorder or a condition belonging to each of these groups.

In some embodiments the compositions, dosage forms and kits of the invention can be applied for the prevention, amelioration and treatment of psoriasis, multiple sclerosis, asthma, fatty liver and major depression.

In some embodiments the compositions, dosage forms and kits of the invention can be applied for the prevention, amelioration and treatment of Inflammatory Bowel Disease (IBD). This type of application has been extensively studied in the EXAMPLES.

The term “Inflammatory Bowel Disease (IBD)” refers herein to various types of disorders generally involving chronic inflammation various parts of the digestive tract. It encompasses herein the two predominant types of IBD, ulcerative colitis (UC) and Crohn's Disease (CD), and further Indeterminate Colitis (approx. 10% of IBD) exhibiting features of both UC and CD, and related and intermediate conditions.

UC and CD encompass herein a range of conditions meeting the diagnostic criteria of IBD. The clinical presentations of UC and CD are often overlapping in terms of symptoms, age of onset, gender distribution and other factors. The differences between UC and CD are predominantly in the location and spread of the inflammation. UC is characterized by a continuous inflammation of the colon and affects only the inner lining. CD on the other hand presents patchy inflammation that can involve all layers of the entire GI tract. The differential diagnosis of UC, CD and Indeterminate Colitis can be performed by endoscopic procedures (e.g., colonoscopy, sigmoidoscopy) and imaging procedures (e.g., X-ray, CT and MR enterographies). Severity of inflammation can be determined by specific severity scores (e.g., Simple Clinical Colitis Activity Index, Crohn's Disease Activity Index) and endoscopic severity scores (e.g., Crohn's Disease Endoscopic Index and Simple Endoscopic Activity Score).

The symptoms UC and CD may range from mild, moderate to severe, depending on the severity and site of inflammation, and are likely to have periodic manifestation with flares of active illness followed by periods of remission.

In some embodiments the compositions, dosage forms and kits of the invention can be used for the prevention, amelioration and treatment of one or more symptoms of UC.

In some embodiments UC can be diagnosed as acute severe ulcerative colitis (ASUC) or inactive or dormant UC.

In some embodiments UC can be diagnosed by the recognized clinical criteria or biomarkers as a mild, a moderate or a severe UC, or a UC in any stage therebetween.

In some embodiments the compositions, dosage forms and kits of the invention can be used for the prevention, amelioration and treatment of one or more symptoms of CD.

Amelioration of IBD or improvement of IBD, and CDI in general, means herein any positive change in disease severity score or a biomarker of disease severity and progression. The present EXAMPLES show how to apply such improvement measures.

In some embodiments an improvement of IBD can comprise a reduction in Simple Clinical Colitis Index (SCCAI) of 3 scores (SCCAI≥3)

In some embodiments an improvement of IBD can comprise a reduction in the levels of the Fecal Calprotectin biomarker (Fcal) of at least 50%.

In some embodiments an improvement of IBD can comprise an improvement in an endoscopic scope of at least 1 (e.g., Mayo endoscopic sub-score).

In some embodiments an improvement of IBD can comprise clinical remission evaluated as SCCAI of less than 2 (SCCAI≤2).

In some embodiments an improvement of IBD can comprise a biomarker remission, for example Fcal of less than 100 μg/g at the end of induction.

In some embodiments an improvement of IBD can comprise a reduction of one or more inflammatory markers, for example a reduction in the C-reactive protein (CRP).

The invention can be further articulated in terms of methods for treating at least one symptom of at least one an CID or an IBD in a subject suffering therefrom, characterized in that the subject is administered a therapeutically effective amount of the compositions, dosage forms and kits of the invention.

The terms “therapeutically effective” or “clinically effective” imply specific doses, amounts or concentrations of actives (e.g., Cur and QD) that confer a therapeutic response, on the level of clinical symptoms or clinically relevant biomarkers (e.g., SCCAI or endoscopic scores, and/or Fcal or CRP biomarkers). This term varies for each active depending on its activity and permeability, and its respective concentration, and further, for each patient depending on patient's specific factors such age, weight, tolerance and compliance. Estimates of therapeutically effective doses or amounts can be derived from clinical trials.

In some embodiments the methods of the invention involve enteral administering of the respective compositions, dosage forms and kits.

In some embodiments the methods can involve administering Cur at a therapeutically effective dose of up to 5000 mg and QD at a therapeutically effective dose of up to 5000 mg, or more specifically Cur at a therapeutically effective dose ranging between 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000 mg and QD at a therapeutically effective dose ranging between 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000 mg.

For specific applications the therapeutically effective doses can be more than 5000 mg Cur and more than 5000 mg QD, or they can range between 5000-5500, 5500-6000, 6000-6500, 6500-7000, 7000-7500, 7500-8000, 8000-8500, 8500-9000, 9000-9500, 9500-10000 mg each, and more.

In some embodiments the methods can involve administering therapeutically effective doses of Cur in the range between about 500 mg and about 3000 mg and QD in the range between about 500 mg and about 3000 mg.

In some embodiments the methods can involve administering therapeutically effective doses of Cur in the range between about 500 mg and about 2000 mg and QD in the range between about 500 mg and about 2000 mg.

In some embodiments the methods can involve administering therapeutically effective doses of Cur in the range between about 500 mg and about 3000 mg and QD in the range between about 500 mg and about 2000 mg.

In some embodiments the methods can involve administering therapeutically effective doses of Cur in the range between about 500 mg and about 3000 mg and QD in the range between about 500 mg and about 1000 mg.

In some embodiments the methods can involve administering therapeutically effective doses of Cur the range between about 500 mg and about 3000 mg and QD in the range between about 1 mg and about 500 mg, or in the range of about 1-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-440, 440-450, 450-460, 460-470, 470-480, 480-490, 490-500 mg.

In further embodiments the methods can involve administering Cur in therapeutic doses as above and almost no QD.

In some embodiments the methods can comprise administering only Cur in therapeutic doses as above.

In some embodiments the methods of the invention can involve administering the compositions, dosage forms and kits at specified periods of time, such as several times a day, every day, every other day, number of times per week, etc., in the course of 1 week, 2 weeks, 3 weeks or 4 weeks or for at least 1 month, 2 months, 6 months, 1 year or more than 1 year or through entire life of the patient.

In some embodiments the methods can involve daily administering of the compositions, dosage forms and kits for at least 1, 2, 3, 4, 5, 6, 7, 8, 9, 10, 11, 12 months, or for at least 2, 3, 4, 5, 6, 7, 8, 9 10 years or through entire life of the patient.

In terms of daily doses, in numerous embodiments the daily dose of Cur can be up to about 5000 mg per day and the daily dose of QD can be up to about 5000 mg per day, or more specifically daily doses ranging between 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000 mg Cur and QD each.

For specific applications the daily doses can be more than 5000 mg Cur and more than 5000 mg QD, or they can range between 5000-5500, 5500-6000, 6000-6500, 6500-7000, 7000-7500, 7500-8000, 8000-8500, 8500-9000, 9000-9500, 9500-10000 mg each, and more.

In some embodiments the daily doses of Cur and QD can be the same.

In other embodiments the daily doses of Cur and QD can be different, depending on the severity of the IBD clinical presentation (also severity of symptoms. For example, in some embodiments, patients with more severe IBD presentation can receive higher doses of QD at the expense of Cur.

In some embodiments, especially during remission and maintenance phases, the daily doses of QD can be tapered to the range between about 1 mg and about 500 mg, or to the range of about 1-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-440, 440-450, 450-460, 460-470, 470-480, 480-490, 490-500 mg.

In some embodiments during remission and maintenance phases, the patient can receive compositions and dosage forms with only Cur, said administering can be simultaneous with or in succession to the compositions, dosage forms or kits of the invention.

The proposed compositions and methods of the invention can be essentially complementary to the existing approaches to the treatment of CID in general, and IBD in particular. In other words, in numerous embodiments the compositions, dosage forms and kits of the invention can be administered together with one or more immune-modulatory or anti-inflammatory drugs or therapeutic agents.

In some embodiments the compositions, dosage forms and kits of the invention can be administered together with or as add-on to one or more anti-inflammatory, immune-modulating agents (e.g., 5-ASA, steroids, azathioprine, 6-MP), biologic drugs (e.g., infliximab, adalimumab, ustekinumab, and vedolizumab), small molecules (e.g., tofacitinib) and/or calcineurin inhibitors (e.g., cyclosporine and tacrolimus).

In some embodiments the compositions, dosage forms and kits of the invention can be administered simultaneously with the concurrent drugs.

In some embodiments they can be administered in succession, before or after, to the concurrent drugs.

In some embodiments the compositions, dosage forms and kits of the invention can be used per se, especially in intractable patients who are resistant or intolerant to the conventional or known treatments.

In other words, in some embodiments the compositions, dosage forms and kits of the invention can be used for treating intractable CID, which is resistant or intolerant to the existing treatments.

More broadly, the compositions and methods of the invention can be used for the prevention, alleviation and treatment of diabetes, cardiovascular diseases, cancer, arthritis, neurodegenerative disorders, psychiatric disorders, autoimmune disorders, inflammatory skin disorders, chronic pulmonary conditions and allergies.

In some embodiments they can be used for the prevention, alleviation and treatment of psoriasis, multiple sclerosis, asthma, fatty liver and major depression.

In some embodiments they can be used for the prevention, alleviation and treatment of IBD.

In some embodiments they can be used for the prevention, alleviation and treatment of UC, including ASUC or inactive or dormant UC, and further a mild, a moderate or a severe UC, or a UC in any stage therebetween.

In some embodiments they can be used for the prevention, alleviation and treatment of CD.

This particular aspect can be further articulated in terms of a combination therapy for treating CID or IBD, or at least one symptom of a CID or an IBD. The concept of combination therapy implies administration of more than one active. The compositions and methods of the invention per se involve a combination of Cur and QD and can further involve one or more additional therapeutic agents.

In other words, the combination therapy of the invention can use any of the aforementioned compositions, dosage forms and kits, and optionally at least one additional therapeutic agent for treating CID or IBD.

In some embodiments the combination therapies can use the same or different therapeutic doses of Cur and QD, as explained above.

In some embodiments the combination therapies can further use compositions and dosage forms with only Cur.

In some embodiments the combination therapies can use daily dose regimens or weekly dose regimens for short or prolonged periods or through the entire patient's life.

One of the distinctive features of the combination therapy of the invention in providing an improved therapeutic effect relative to the effect of the same amounts of Cur and QD when used alone and a reduced adverse effect relative to the same amount of QD alone. This particular property has been previously articulated.

In some embodiments the combination therapy can provide therapeutic effect that is synergistic relative to the effect of the same amounts of Cur and QD when used alone. In other words, the effect of the therapy can be 2-fold to 10-fold larger than the effects of Cur and QD alone, or in the range between about 2-3 fold, 3-4 fold, 4-5 fold, 5-6 fold, 6-7 fold, 7-8 fold, 8-9 fold and 9-10 fold larger than the effects of the Cur and QD alone.

The issue of combination therapy has become especially attractive in view of recent findings by the inventors, showing that the effect of CurQD is mediated by activation of the AhR signaling pathway and CYP1A1, and that this effect is unique and specific and was not observed with other conventional medications or biologics. The implications of these findings are two-fold:
1. As CurQD acts by an alternative molecular mechanism, it can provide an effective supplementary therapy to the existing conventional and the new-generation therapies of IBD;
2. As CurQD activates AhR and specifically CYP1A1, its curative effect can be modulated by additional type of ligands acting on the AhR signaling pathway.

To this last point, it has been known that AhR is activated by a wide range of synthetic and natural ligands, including among others arachidonic acid (ARA), pyrene, and tryptophan and flavonoid derivatives. In other words, the curative effect of CurQD on CID and IBD in particular can be further enhanced or improved when co-administered on the background of certain types of supplements and diets, such as ARA supplements and ARA-rich diets (e.g., meat, poultry, eggs, fish and dairy foods), or L-tryptophan supplements and tryptophan-rich diets (e.g., chicken, turkey, red meat, pork, tofu, fish, beans, milk, yogurts, nuts, seeds, oatmeal, and eggs), or flavonoid supplements (e.g., quercetin, silymarin, green tea extracts, and rutin) or flavonoid-rich diets (e.g., tea, citrus fruit, citrus fruit juices, berries, red wine, apples, and legumes).

CYP1A1, in turn, is responsible for the metabolism of numerous toxins and drugs, including pro-carcinogens and certain anti-carcinogens and other drugs. It also participates in the metabolism of steroidal hormones such as estrogens. In other words, CurQD should be contraindicated on the background of certain drugs, and specifically drugs metabolized by the CYP1A1 enzyme. Alternatively, it can be especially effective when co-administered on the background of drugs activating the expression of CYP1A1.

In addition, it has been known that diet have close relationship with the composition and maintenance of a healthy microbiome in the gut. It is therefore conceivable that the effect of CurQD can be further enhanced or improved when co-administered on the background of certain microbiota derived AhR ligands that are comprised in many probiotics, prebiotics and postbiotics supplements.

Therefore, the invention can be further articulated in terms of combination therapies and methods comprising enteral administering of the compositions, dosage forms or kits of the invention together with certain type of nutraceuticals, supplements and/or a diets comprising one or more ligands of the Aryl Hydrocarbon Receptor (AhR) or activators of the AhR signaling pathway.

In some embodiments such AhR activators can be arachidonic acid-(ARA), tryptophan- or flavonoid-rich nutraceuticals, supplements and/or diets.

In some embodiments the combination therapies and methods of the invention can involve concomitant use of enteric formulas, yogurts, vitamins, prebiotics, oligosaccharides, probiotics or other microbiota-altering agents.

In some embodiments diet can be selected from Mediterranean Diet, CD Exclusion Diet (CDED), UC Exclusion Diet (UCED), Specific Carbohydrate Diet (SCD), Anti-Inflammatory Diet (AID), CD-TREAT Diet, or any other diets that specifically prescribed in the context of IBD.

Ultimately, the invention can be articulated in terms of use of a combination of Cur and QD, or an extract of any part of a plant producing Cur or QD, wherein the Cur and QD are present in a weight ratio ranging between 100,000:1 and 1:100,000, respectively, for the manufacture/preparation of a medicament or a nutraceutical with improved therapeutic effect and reduced adverse effects. The term “about” herein denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

The figures and the experimental examples provided herein serve only illustrative purposes and are not intended to be limiting as the scope of the invention will be limited only by the scope of the appended claims.

### Example 1

Case Study 1: The Effect of CurQD Combination in a 24-Years Male with UC

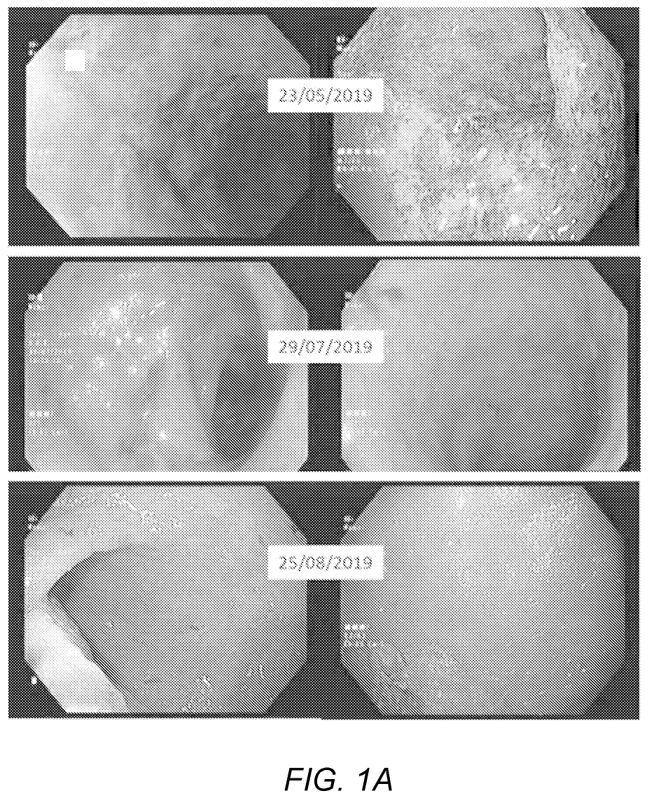

Patient 1, a 24 years-old male, was presented with extensive acute UC refractory to cyclosporine which persisted for two years and with a prior prolonged hospitalization. He was improved with infliximab, which was later optimized due to partial response on 7 mg/kg infliximab every 4 weeks and 100 mg/day 6-mercaptoputine (6-MP). He had daily 6-7 bowel-movements, half of which with blood. Infliximab levers were 11.9 mcg/ml without anti-drug antibodies. CRP was nine-times the upper normal limit. Fcal was 670 mg/gr. Sigmoidoscopy showed Mayo 3 inflammation up to 30 cm from the anal verge (FIG. 1a). Infectious workup was negative. Swapping biologic was considered but the patient opted for short-term trial with the CurQD therapy.

CurQD therapy was initiated with daily administration of 1000 mg Cur and 1500 mg QD (EviNature, Binyamina, Israel) provided as add-on to infliximab and 6-MP. Bleeding ceased within 10 days and complete remission was observed within several weeks. Two follow-up lower endoscopies performed at 8 and 12 weeks after CurQD initiation showed marked endoscopic improvement to Mayo 1 mucosal appearance on the later examination (FIG. 1a). The patient was maintained on the same dose of infliximab and 6MP and with gradual tapering of QD dose to 500 mg on every-other-day. The clinical and endoscopic flare which emerged 3 months later was treated by increasing QD dose to 1000 mg/daily, after the flare the dose was reduced to 500 mg/day. Eight months later, while in complete remission, the patient stopped infliximab. He has since remained in complete remission on CurQD and 6MP until his last follow-up 31 months after starting the CurQD therapy. No adverse events were recorded, and a cardiac echocardiogram performed at 16 months on the CurQD therapy was normal.

### Case Study 2: The Effect of CurQD Combination in a 56-Years Female with UC

Patient 2, a 56 years-old female, was diagnosed with extensive active UC a year earlier, which was refractory to maximal oral and topical mesalamine therapy and did not improve with 9 mg/daily budesonide-MMX. She was presented with 4-5 bloody bowel movements a day, weakness and abdominal pain, hemoglobin level at 10.3 g/dl, CRP was 6 times of upper normal limit and sigmoidoscopy showing severely ulcerated Mayo 3 grade inflammation up to 45 cm (FIG. 1b). She resisted biologic medications (vedolizumab) and consented to a short-term CurQD therapy.

After initiation of the CurQD treatment, rectal bleeding and increased bowel movements resolved rapidly. Follow-up sigmoidoscopy at 7 weeks showed marked improvement in mucosal appearance and a repeat sigmoidoscopy at 5 months showed mucosal scarring and complete healing (FIG. 1b). Since then, the patient has been maintained on 3 gr/day mesalamine and CurQD, 500 mg/every-other-day QD and with 1000 mg/daily Cur, for 49 months until the last follow-up visit. Two flares observed during this period (diagnosed by endoscopic inflammation and Fcal elevation) were treated by temporary increasing QD dose to 1000 mg/daily and tapering back 500 mg to every alternating-day. Echocardiogram findings at 15 months were unremarkable.

### CONCLUSIONS

Both these case studies prompted the inventors to continue investigating the feasibility of oral CurQD therapy for UC in structured and controlled clinical trials.

### Example 2: Prospective Open-Label Phase 1 Clinical Trial on Oral CurQD Therapy

### 2.1 Study Group

Patients (N=10) aged>18 years-old were recruited to the study. Eligibility criteria included active UC (SCCAI≥5) and active colonic inflammation (modified Mayo endoscopic sub-score≥2 in the extended proximal to the rectum (>15 cm) at the screening lower endoscopy). Concomitant medications were allowed prior and during the study, including biologics (if received) maintained at a stable dose for 4-12 weeks prior to inclusion, and/or corticosteroids maintained at ≤20 mg/day of prednisone or equivalent for at least 2 weeks prior to inclusion. Exclusion criteria included pregnancy, allergy to Cur or QD, active infection (either enteric or elsewhere), patients with uncontrolled renal, liver, lung or cardiovascular disease, hypertension, diabetes, migraine or neurological disease, chronic pancreatitis or gallstone disease, and patients with significant laboratory abnormalities (e.g., anemia<10 mg/dl, leukopenia WBC<4000/mcl, thrombopenia <100,000/ml, abnormal coagulation tests or elevation of liver or kidney function tests). Patients who have never received any treatment for UC were also excluded.

### 2.2 Tested Items

The tested dosage forms included Cur and QD oral capsules with enteric coating (Eudraguard Biotic polymer PH-dependent coated HPMC capsules) with 500 mg herbal QD extract in the form of dry powder or 500 mg Cur as dry powder (both GMP grade provided by EviNature, Israel).

### 2.3 Clinical Workup

Clinical screening included physical examination, blood tests for complete blood count, liver and kidney function tests, CR, and coagulation tests. Liver ultrasonography and cardiac transthoracic echocardiograms were performed before enrollment. Stool tests for enteric infections and for determining Fcal levels and sigmoidoscopy with biopsies were performed. The endoscopic Mayo sub-score was determined separately by two blinded investigators.

### 2.4 Study Protocol

Patients received open label daily treatment with Cur and QD oral capsules (n=3), i.e., 3 gr total CurQD daily dose (3×500 mg Cur and 3×500 mg QD), for 4 weeks. Adverse events assessed on day 3 via telephone interview and clinical outcomes after 4 weeks, with the measured primary outcomes as the percentage of patients with significant clinical response (reduction of SCCAI≥3), significant biomarker response (>50% Fcal reduction) and clinical remission (SCCAI≤2) at week 4.

### 2.5 Results

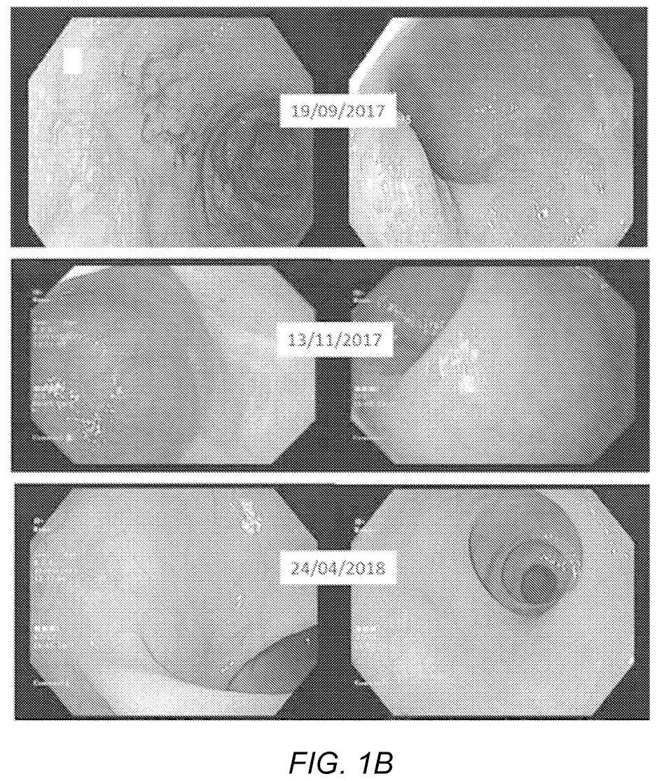

Significant proportion of patients improved with the oral CurQD therapy as per the measurements of SCCAI and Fcal, FIGS. 2a and 2b, respectively. Specifically, about 60% of the patients (7 out of 10) showed significant clinical response (reduction of SCCAI≥3), about 40% (4 out of 10) showed significant biomarker response (>50% reduction of Fcal) and about 30% (3 out of 10) achieved clinical remission (SCCAI≤2).

### 2.6 Conclusions

This study fulfilled the preliminary requirement of achieving at least one primary outcome in at least 30% of patients without severe adverse events, thus prompting to continue investigating the safety and efficacy of oral CurQD therapy in a double-blind placebo-controlled clinical trial.

### Example 3: Double-Blind Placebo-Controlled Clinical Trial on Oral CurQD Therapy

### 3.1 Study Group

Patients' recruitment and eligibility criteria were as above (N=42) 3.2 Tested items

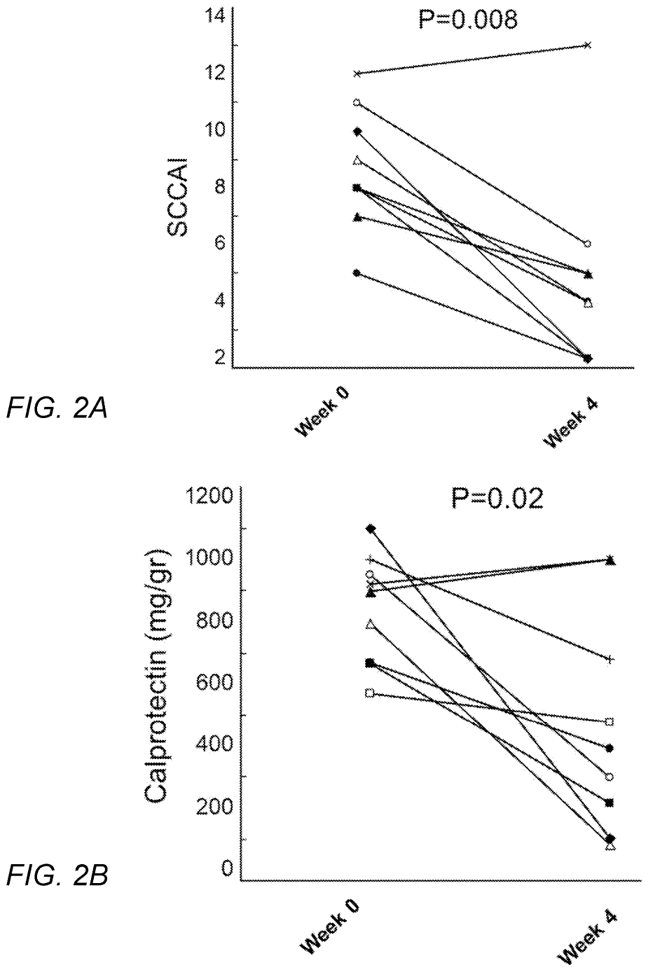

The tested dosage forms included enteric-coated Cur and QD oral capsules as above (each 500 mg QD or 500 mg Cur) and analogous placebo capsules (FIG. 3).

### 3.3 Clinical Workup

Clinical screening was conducted using clinical parameters as above.

### 3.4 Study Protocol

Patients were blinded to CurQD and placebo, receiving daily treatment with Cur and QD oral capsules (n=3 as above) or placebo capsules (n=3) for 8 weeks. Adverse events and clinical outcomes were assessed as above at week 8.

### 3.5 Results

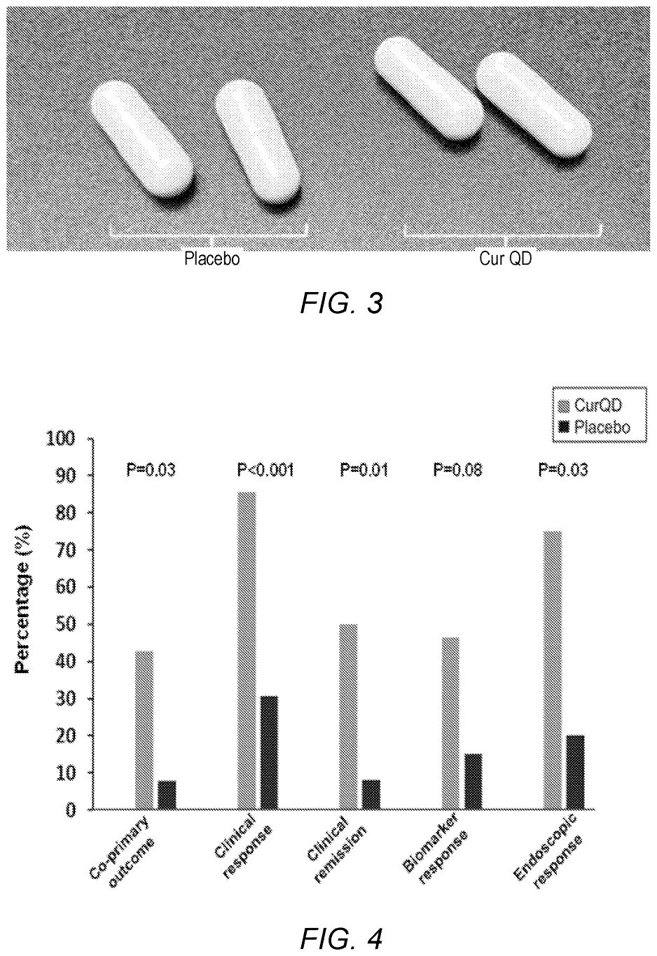

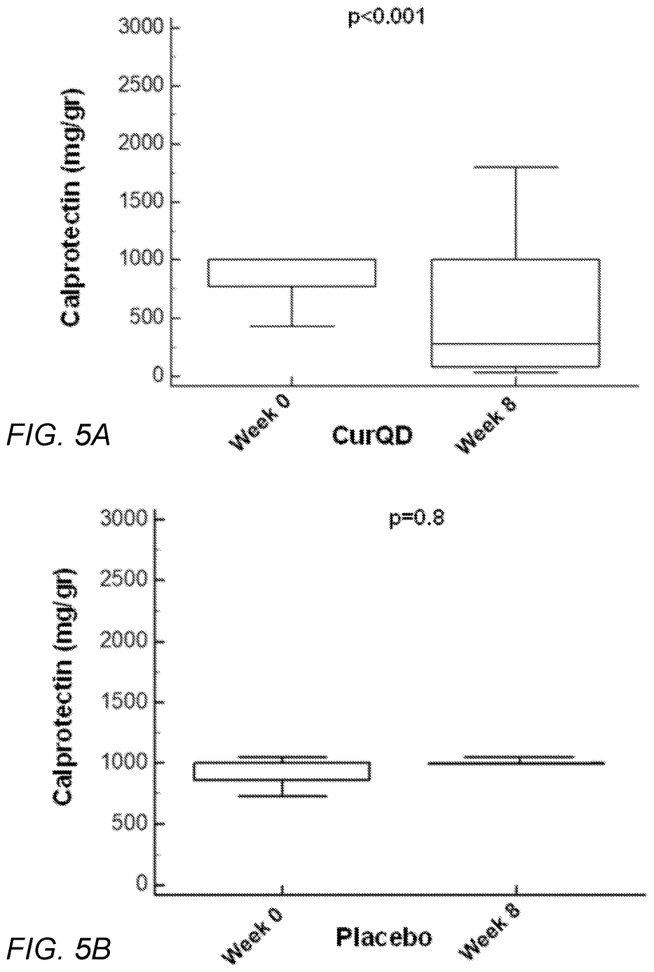

Preliminary analysis of the study results showed significant improvement of UC by a number of parameters in the CurQD treated group vs. the placebo group. Specifically, the co-primary outcome was achieved in 43% and 8% patients (p=0.033), clinical response in 85.7% vs. 30.7% patients (p<0.001), endoscopic improvement in 75% vs. 20% patients (p=0.036), >50% Fcal reduction in 46.4% vs. 15.4% patients (p=0.08) and clinical remission in 50% vs. 8% patients (p=0.01) in the CurQD and placebo groups, respectively (FIG. 4). Analysis of specific effects of the CurQD therapy on Fcal levels showed that the median Fcal levels were significantly reduced by week 8 in the CurQD group (FIG. 5a) vs. the placebo groups (FIG. 5b) (p<0.001). Regarding adverse events, worsening UC was observed in 14.3% (4/out of 28) patients in the CurQD group vs. 46.2% (6 out of 13) patients in the placebo group (p=0.048). In addition, one male 19 years-old patient on CurQD experienced asymptomatic elevation of liver transaminases. In all these cases the CurQD therapy was terminated.

### 3.6 Conclusions

Both these studies strongly supported the prospect of applicability of the oral CurQD therapy as add-on or a replacement therapy for UC and potentially for other IBDs.

### Example 4: Advanced Dosing Protocol for Administration of Oral CurQD Therapy

More recently, the inventors developed an advanced protocol for administration of oral CurQD therapy to maximize therapeutic efficacy while minimizing potential side effects of QD. Specifically, CurQD is initially (e.g., first 4-8 weeks) given with higher daily doses of QD vs. Cur (e.g., 1-1.5 gr/day QD and 1-2 gr/day Cur), after which the QD doses are gradually tapered (e.g., every two weeks) to lower doses of QD vs. Cur (e.g., to 1.2, 1, 0.8, 0.6 and 0.5 gr/day QD and 2, 2.2 and 2.5 gr/day Cur), ending with only Cur (e.g., 2-3 gr/day Cur) as long-term treatment for maintaining remission. Upon emergence of clinical flare, the protocol is converted to a short-term CurQD dosing Cur (e.g., 1-1.5 gr/day QD and 1-2 gr/day Cur) for 4-10 weeks. This protocol is currently tested in a controlled study of patients with active UC.

### Example 5: a Retrospective Multicenter Cohort Study of Adult UC Patients

Additional support for the success of oral CurQD therapy in the treatment of UC was provided by a more recent retrospective multicenter cohort study gathering the data from five academic centers (2018-2022). Curcumin and QingDai (QD, Indigo) were shown effective for treating active ulcerative colitis (UC). We aimed to evaluate the real-world experience with the herbal combination of Curcumin and QD (CurQD) to induce remission in active UC.

### 5.1 Methods

Data on patients with active UC was collected from medical records, including indices of UC diagnosis (SCCAI score), biomarkers levels (fecal calprotectin (FC) and C-reactive protein (CRP) levels), use of CurQD and other therapies (corticosteroids or biologics) and the record of adverse events. Active UC was defined as SCCAI score ≥3. The primary endpoint was clinical remission at 8-12 weeks, defined as SCCAI≤2 and a decrease ≥3 points from baseline. Secondary endpoints were clinical response (SCCAI decrease ≥3 points), corticosteroid free-remission, FC response (decrease ≥50%), and normalization (FC≤100 μg/g), and safety (no escalations or hospitalizations during the induction period). The study included 88 patients, about 49% of the patients were biologic/small molecules experienced and 39% received more than 2 biologics/small molecules. Oral CurQD therapy was used alone or as an add-on to the existing therapies. Patients received daily doses of 1-2 gr QD and 1-3 Cur as dry powders. Patients with more severe active UC received higher QD at the expense of Cur.

### 5.2 Results

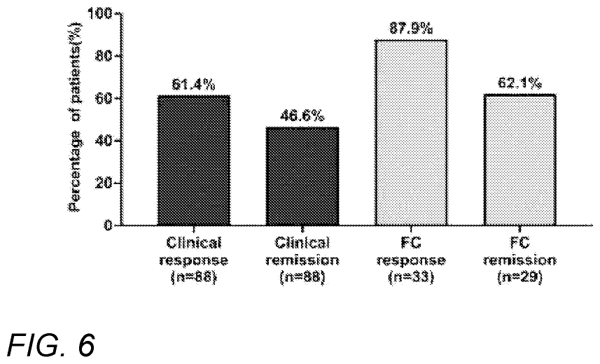

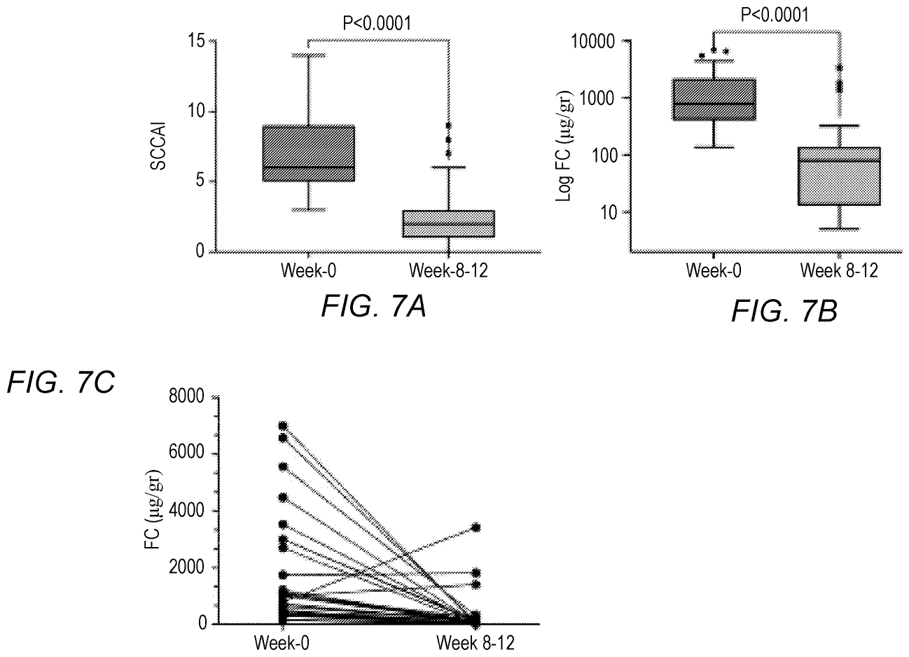

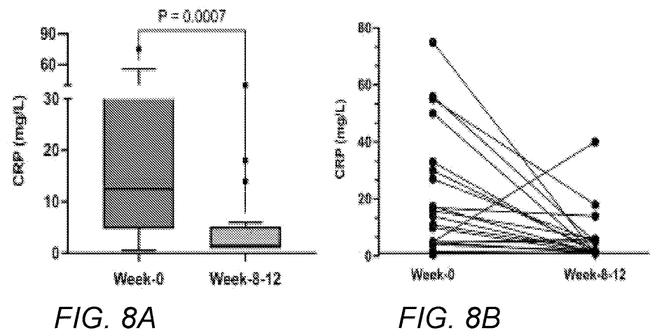

The results of the study are summarized in FIG. 6, information on changes of specific indices and markers is provided in FIGS. 7a-7c and FIGS. 8a-8b. Overall, out of patients included in this study (n=88), clinical response was achieved in 61.4%, remission in 46.4%. Median SCCAI decreased from 7 (IQR 5-9) to 2 (IQR: 1-3), p<0.0001. Of the 26 patients on corticosteroids at baseline, 7 (26.9%) achieved corticosteroid-free remission. Median FC decreased from 730 μg/g (IQR: 384-1195) at baseline (n=45) to 78 μg/g (IQR: 12-141) at the end of inductions (n=38), p<0.0001. FC response and normalization were achieved in 29/33 (87.9%) and 18/29 (61.2%), respectively. Among 43 biologic/small molecule experienced patients, clinical response was achieved in 58.1% and clinical remission in 39.5%. No overt safety signals emerged.

### 5.3 Conclusions

This experience in a representative cohort of adult patients with active UC showed that oral CurQD therapy was effective in inducing remission by clinical indices of UC and biomarkers, as an only therapy and as an add-on therapy.

### Example 6: a Retrospective Cohort Study of Pediatric UC Patients

An additional study evaluated the efficacy of oral CurQD therapy in pediatric patients with mild-moderate active UC who failed to respond to conventional therapies.

### 6.1 Methods

The study included children (age≤18) with mild-to-moderate active UC who were treated with CurQD between 2017 and 2021. Clinical, laboratory and endoscopic data were retrieved from medical records. Disease was assessed by the Pediatric UC Activity Index (PUCAI), physician global assessment (PGA), and by Fecal Calprotectin (FC) levels and endoscopic MAYO scores prior to and after treatment. The primary outcome was clinical response after induction (up to 16 weeks from treatment initiation), defined as improvement of PUCAI by 10 points. Discontinuation of CurQD due to adverse events or lack of response, hospitalization or reversal to biologics or corticosteroids were excluded from the study. The study included 30 patients (18 males, 60%), age 12.17±3.89 years. Disease diagnosis was pancolitis in 15 (50%), left sided colitis in 13 (43%), and proctitis in 2 (7%) patients. Eighteen patients (60%) were naïve to biologic treatment, and 7 (22%) received more than one biologic therapy. At CurQD induction, six patients (19%) were treated with corticosteroids, 9 (28%) with biologics, and 13 (40%) with 5-ASA only. The daily CurQD dose was 500-3000 mg QD with 1000-4000 mg CurQD.

### 6.2 Results

Final analysis of this study showed that the mean PUCAI decreased from 31.3±12 at baseline to 10.9±8.8 at the end of induction (week 11.6±3.5, range 8-16) (p<0.001). FC decreased from a median of 749 (intequartile range (IQR) 566-1000) to 39 (IQR 12-132) at the end of induction (n=15, p=0.04). PGA-based clinical remission was observed in 17 (71%), clinical response in 4 (16.5%) of the patients at the end of induction, and no response in 3 (12.5%) patients. Adverse events as headache or hematuria were observed in 2 patients, which were resolved after CurQD discontinuation, and elevated transaminases during maintenance in 2 patients. The mean follow up was 14.5±11 months. Ten patients (32%) flared while on CurQD treatment, one of them regained remission with no treatment change, one regained response with increased CurQD dose, and in 7 patients the treatment was discontinued. Overall, out of 18 patients with available data, 10 were in remission, 2 were in clinical response and 6 had an active disease.

### 6.3 Conclusions

Oral CurQD therapy was found effective and safe as add-on treatment in pediatric patients with mild to moderate UC in providing an adequate response and remission in the majority of the study patients even after failure of biological therapy.

### Example 7: CurQD Combinations Containing Various Types QD Extracts

The inventors further explored the boundaries of the effective Cur and QD concentrations that could confer to the present compositions improved therapeutic effects and reduced adverse effects of QD. According to Chinese Pharmacopoeia, an effective QD preparation should contain at least 2% Indigo and at least 0.13% Indirubin (Qi-Yue Y et al in Chin Med 2020, 15 (1): 127). Elevated concentrations of the two actives have been related to a range of adverse effects, including among others pulmonary hypertension.

### 7.1 Methods

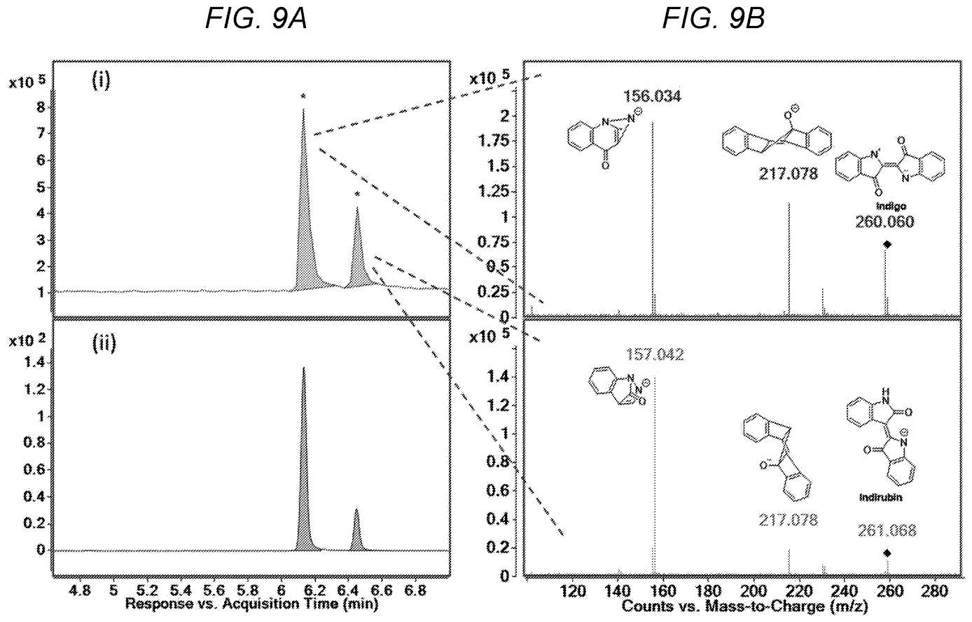

Different types of QD extracts with different concentrations of Indigo and Indirubin were used to construct CurQD combinations with daily therapeutic doses of Cur and QD 1:1. The relative content of Indigo and Indirubin were determined using chromatographic methods (FIGS. 9a-9b). Patients with active UC were treated with different CurQD preparations using the regular treatment protocol, a part of the patients were treated using a temporally tailored protocol, under a tapered dose regimen of QD. Pulmonary hypertension (elevation of pulmonary vascular pressure), a known QD side effect, was monitored by echocardiography.

### 7.2 Results

Testing the therapeutic efficacy of various CurQD compositions by a number of IBD parameters, the study showed that compositions with relatively low content of Indigo and Indirubin, minimum 0.55% and 0.06% (w/w extract) respectively, still retained adequate clinical efficacy. The results are shown in Table 1 below.

TABLE 1
Clinical efficacy of CurQD compositions
with content of QD actives

| BATCH NUM | INDIGO | INDIRUBIN | CLINICAL EFFICACY |
|---|---|---|---|
| B1 | 6.04% | 0.25% | Yes |
| | 5.78% | 0.27% | |
| B2 | 1.40% | 0.127% | Yes |
| | 1.38% | 0.128% | |
| B3 | 1.82% | 0.12% | Yes |
| | 1.33% | 0.087% | |
| B4 | 1.43% | 0.08% | Yes |
| | 1.27% | 0.087% | |
| B5 | 1.06% | 0.067% | Yes |
| | 0.55% | 0.062% | |
| B6 | 0.00% | 0.000% | No |
| | 0.00% | 0.000% | |

Furthermore, using the B5 preparation together with the tapered dosing protocol (tapered QD dose), further improved the safety profile of the CurQD combination, suggested by and total absence of pulmonary hypertension events and neither any elevation of pulmonary vascular pressure in the CurQD treated patients as opposed to the known rate of pulmonary hypertension of 1:90-100 in patients treated by QD alone (Naganuma, J in Gastroenterol 2019 54 (10): 891-896).

### 7.3 Conclusions

This study suggested that the oral CurQD therapy is sufficiently effective to permit reduced the concentrations of Indigo and Indirubin, the two actives responsible for the majority of QD related side effects, below the critical risk conferring threshold.

### Example 8: Effect of CurQD on Activation of AhR Pathway

The inventors have further explored the mechanism of action of CurQD and its downstream effectors. Several previous studies suggested that the QD component (Indigo and Indirubin) exert agonistic effect on the aryl-hydrocarbon receptor (AhR) thereby activating the AhR signaling pathway and inducing a reparative effect in a mouse model of colitis. AhR is a ligand-activated transcription factor that ultimately acts in the nucleus in promoting transcription of various genes, including among others cytochrome P450 1A1 (CYP1A1) which is one of the most important xenobiotic-metabolizing enzymes and an important regulator of immune response in the intestine. Therefore, the inventors studied the expression of CYP1A1 in samples of colonic mucosal tissue obtained from UC patients, using RT-PCR.

### 8.1 Methods

Samples of colonic tissue were obtained during lower endoscopy from UC patients treated with CurQD and patients treated by other medications or placebo. Tissue specimens were immersed in liquid nitrogen. Biopsy samples were lysed and homogenized in TRI-Reagent and extracted according to the manufacturer's instructions (Life technologies, USA). Reverse transcription was performed on lug total RNA using the High Capacity RNA to cDNA kit according to the manufacturer's instructions (Applied Biosystems Foster City, CA USA) and Hs01054796_g1 assay for CYP1A1 and Hs99999905_m1 assay for GAPDH (housekeeping gene).

### 8.2 Results

The results of these experiments are summarized in FIGS. 9a-9b. Figures show that the levels of CYP1A1 were up-regulated in the tissues of patients treated with CurQD as opposed to the treatments with conventional therapies, e.g., 5ASA and biologics, and placebo. The expression of CYP1A1 in rectal mucosa significantly increased from the median of 13.9 (15-75% IQR 2.8-29.6) at baseline to 334 (IQR67.2-2922, n=11, p=0.006) at week 8 following CurQD treatment, while it did not change with placebo (1.55, 0.73-2.4 versus 2.2, 0.9-3.5, before and after placebo, respectively, n=2, p=0.7 because of small sample size). CYP1A1 upregulation was more pronounced in patients who had a better response to CurQD (mucosal healing with Mayo sub-score 0/1) than in CurQD-treated patients without mucosal healing (1600, IQR 213-2967, n=8 versus 14, IQR1.9-1513, n=5, p=0.17 because of small sample size). When comparing CYP1A1 expression in the tissues of patients treated by other medications, the expression levels of CYP1A1 in patients who achieved mucosal healing after CurQD treatment was significantly higher than in analogous patients after treatment with biologics (anti-TNFs and vedolizumab) or with 5ASA (1600, IQR 213-2967, n=8 versus 2.1, IQR0.3-15, n=5, p=0.002, and versus 4.6, IQR1.6-16.4, n=4, p=0.004, respectively). This result was highly significant, despite the small sample size, and surprising.

### 8.3 Conclusions

Overall, it was found that the effect of CurQD is mediated by activation of the AhR signaling pathway and its downstream effectors, and that this effect is unique and specific and was not observed with other conventional medications or biologics. These results further suggest that CurQD constitutes a novel and alternative treatment for IBD, and due to the fact that it acts by a different molecular mechanism than the other known drugs, it can provide alternative and complementary methods of treatment of for this highly burdensome group of disorders, and for adult and pediatric UC in particular.

1. A composition comprising a combination of Curcumin (Cur) and Qing-Dai (QD), or an extract of any part of a plant producing Cur or QD, wherein the Cur and the QD are present in a weight ratio ranging between 10:1 and about 1:10, respectively, and
thereby providing an improved therapeutic effect relative to the effect of the same amounts of Cur and QD when used alone and/or a reduced adverse effect relative to the same amount of QD alone.

2. The composition of claim 1 which is formulated for enteral, sublingual or buccal administering.

3-4. (canceled)

5. The composition of claim 2 which is an oral composition.

6-9. (canceled)

10. The composition of claim 2, wherein the Cur and/or the QD are in powder, liquid or liquid gel forms.

11-12. (canceled)

13. The composition of claim 2, wherein the composition is formulated as a suppository or an enema.

14. The composition of claim 2, wherein the oral composition is the form of a tablet, a pill, or a capsule with or without an enteric coating.

15. (canceled)

16. A dosage form comprising the composition of claim 1.

17. (canceled)

18. The dosage form of claim 16, wherein the amount of Cur is in the range between about 500 mg and about 3000 mg and the amount of QD is in the range between about 500 mg and about 3000 mg.

19. (canceled)

20. The dosage form of claim 16, wherein the amount of Cur is in the range between about 100 mg and about 500 mg and the amount of QD is in the range between about 1 mg and about 500 mg.

21-22. (canceled)

23. A kit comprising:
the dosage form of claim 16,
a dosage form comprising Cur in an amount of up to about 5000 mg, and
instructions for use.

24-31. (canceled)

32. A method of preventing, ameliorating, or treating at least one symptom of a chronic systemic inflammatory disorder or condition (CID) in a subject suffering therefrom, comprising enteral administering to the subject a therapeutically effective amount of a composition or a dosage form comprising a combination of Cur and QD, or an extract of any part of a plant producing Cur or QD wherein the Cur and the QD are present at a weight ratio ranging between 10:1 and about 1:10 of the combination, respectively; the method optionally further comprising enteral administering to the same subject a composition or a dosage form comprising a therapeutically effective amount of Cur, said second administering of Cur being simultaneous with or in succession to the first administering of the Cur and QD combination.

33-34. (canceled)

35. The method of claim 32, wherein the CID comprises an Inflammatory Bowel disease (IBD).

36. The method of claim 35, wherein the IBD comprises an Ulcerative Colitis (UC) or Crohn's Disease (CD).

37. The method of claim 36, wherein the UC comprises an Acute Severe UC (ASUC) or inactive or dormant UC.

38. (canceled)

39. The method of claim 32, wherein the composition or the dosage form with the combination of Cur and QD is administered to the subject daily for a period of at least 1 month, 2 months, 6 months, 1 year or more than 1 year, or through entire life of the patient.

40. The method of claim 32, comprising further administering to the subject at least one additional therapeutic agent for the treatment of CID, simultaneously with or in succession to the composition or the dosage form with the Cur and QD combination.

41. (canceled)

42. The method according to claim 32, further comprising administering to the subject a nutraceutical, a supplement and/or a diet comprising one or more ligands of the Aryl Hydrocarbon Receptor (AhR) or activators of the AhR signalling pathway.

43. The method of claim 42, wherein said nutraceutical, supplement and/or diet are selected from arachidonic acid-(ARA), tryptophan- or flavonoid-rich nutraceuticals, supplements and/or diets.

44. The method of claim 43, wherein said nutraceutical or supplement are selected from enteric formulas, yogurts, vitamins, prebiotics, oligosaccharides, probiotics or other microbiota-altering agents.

45. The method of claim 43, wherein said diet is selected from Mediterranean Diet, CD Exclusion Diet (CDED), UC Exclusion Diet (UCED), Specific Carbohydrate Diet (SCD), Anti-Inflammatory Diet (AID), CD-TREAT Diet or another diet specifically prescribed for IBD.

46-55. (canceled)
